(19) United States
(12) Patent Application Publication
(10) Pub. No.: US 2008/0154180 A1
(43) Pub. Date: Jun. 26, 2008

(54) Ultra-Lipodissolve Method

(76) Inventor: Walter Ray Seidel, Ruidoso, NM (US)

Correspondence Address: PEACOCK MYERS, P.C., 201 THIRD STREET, N.W., SUITE 1340, ALBUQUERQUE, NM, 87102, US

(21) Appl. No.: 11/957,990

(22) Filed: Dec. 17, 2007

Related U.S. Application Data
(60) Provisional application No. 60/870,219, filed on Dec. 15, 2006.
(60) Provisional application No. 60/890,072, filed on Feb. 15, 2007.

(51) Int. Cl. A61N 7/00; A61N 1/30

(52) U.S. Cl. 604/21; 604/22

(57) ABSTRACT

A method for destroying fatty tissue using a lypo-lysis agent or adipose emulsifier.

### CROSS-REFERENCE TO RELATED APPLICATIONS

This application claims priority to and the benefit of U.S. Provisional Application Ser. No. 60/870,219, entitled “Ultra-Lipodissolve Method”, to Walter Ray Seidel, filed on Dec. 15, 2006, and the specification thereof is incorporated herein by reference. This application relates to U.S. Provisional Application Ser. No. 60/890,072, entitled “Ultra-Lipodissolve Method”, to Walter Ray Seidel, filed on Feb. 15, 2007, and the specification thereof is incorporated herein by reference.

### BACKGROUND OF THE INVENTION

1. Field of the Invention (Technical Field)

The present invention relates to methods for non-surgical destruction of fatty tissue.

2. Description of Related Art

### BRIEF SUMMARY OF THE INVENTION

The present invention is of a method to destroy fatty tissue in a human. This method injects patients with a lipo-lysis agent, such as phosphatidylcholine (PC) and deoxycholate (DC), in order to destroy fatty tissue in a patient's body. After an injection, massage or ultrasound is used on the injection site to actively diffuse the lipo-lysis agent within the fatty tissue, which thereby improves the final area with much reduced nodularity. The massage and/or ultrasound is preferably done every 14 days unless nodules are noted, in which case the next lipo-lysis agent injection would be postponed for another week for further examination.

The present invention is a method for destroying fatty tissue by receiving mono-polar radio-frequency destruction treatment, administering an injection of a lipo-lysis agent into fatty tissue at least once, and performing a skin tightening procedure on the injection site. The mono-polar radio-frequency destruction treatment preferably raises the temperature of the intended treatment area to at least 43° C. for approximately two minutes. The method can also include ultrasounding the treated area to actively diffuse the lypo-lysis agent within the fatty tissue. The ultrasounding is preferably performed at 1 to 3 Hertz for at least 10 minutes. The method can alternatively include massage to the treated area to actively diffuse the lypo-lysis agent within the fatty tissue.

The lypo-lysis agent is preferably administered every 14 days. The injection of lypo-lysis agent is preferably 0.8 to 1.0 cc into fatty tissue and each injection of the lipo-lysis agent is preferably at least 2 cm away from the previous injection.

The skin tightening procedure is preferably performed using bi-polar radio-frequency, broadband light, or both.

In another preferred embodiment, the lypo-lysis agent is an adipose emulsifier.

Objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims (if any).

### DETAILED DESCRIPTION OF THE INVENTION

The present invention is of a method for destruction of adipose tissue, referred to herein as Ultra-LipoDissolve™. The treatment cycle for Ultra-LipoDissolve is preferably 14 days and can be repeated every 14 days as necessary. In a preferred embodiment, the part of the body receiving the treatment first receives a mono-polar radio-frequency destruction treatment that raises the temperature of the treatment site to a minimum of approximately 43° C. for at least approximately 2 minutes. The treated area is then thoroughly cleaned with an antiseptic and then injected with a lipo-lysis agent. The injection is preferably within the adipose tissue. A non-limiting example of a lipo-lysis agent is phosphatidylcholine deoxycholate. The amount of lipo-lysis agent being injected in any site with the exception of the face and neck is preferably approximately 0.8 to 1.0 cc per injection site. Injection sites are preferably at least approximately 2 cm apart from each other.

Following the injections, ultrasound and/or massage therapies are preferably applied to the area to cause active diffusion of the lipo-lysis agent within the adipose tissue, which thereby improves the final area with much reduced nodularity. Ultrasound is preferably administered between approximately 1 and 3 Hz to the injection site area for a minimum of about 10 minutes. Massage is preferably done every 14 days unless nodules are noted, in which case the next lipo-lysis agent injection would be postponed for another week for further examination.

After approximately 7 days following the injection of the lypo-lysis agent, a skin tightening procedure is preferably performed over the treatment area and more preferably performed over an area approximately 50% larger than the treatment area. The skin tightening treatment facilitates enhanced tissue retraction and further enhances destruction of the underlying adipose tissues and cellulite. Skin tightening is preferably performed using either a bi-polar radio-frequency device or a broadband light device or both.

The invention can also be used on the face and neck. Preferably, a lipo-lysis agent injection employs a similar protocol with the exception of dosing of the lipo-lysis agent which is preferably adjusted appropriately for the site and volume of adipose tissue located on the face and neck.

The protocol for Weeks 2 through 6 is preferably adjusted based on clinical examination, findings, and how the patient is tolerating the procedure for any given treatment zone. The neck and face preferably has less required treatments, but again is preferably adjusted based on the site and volume of the adipose tissue.

In another preferred embodiment, the first week of treatment comprises injection of an adipose emulsifier (AE). The injections of the AE in any site is preferably 0.2 to 1.0 cc per injection site as clinically indicated by site and volume of the adipose tissue as well as how the patient tolerates the procedure. The injection sites are preferably spaced apart as clinically indicated. Following the injections, ultrasound and/or massage is preferably applied to the area in order to cause active diffusion of the AE, which improves the final area with much reduced nodularity. This preferred embodiment protocol is performed approximately every 14 days as clinically indicated unless nodules are noted. If nodules are noted, then the next AE injection is preferably postponed as clinically indicated after further examination.

On the off week (i.e., those with no injections of the AE), the treated area would preferably be followed up with a light base skin tightening procedure and a radio frequency (RF) procedure with a Unipolar or Bipolar unit (skin tightening and adipose tissue destruction) for further enhanced destruction of the underlying adipose tissues and cellulite as well as skin tightening as clinically indicated.

Treatment of the face and neck with an AE injection preferably has a similar protocol to the above preferred embodiment with the exception of dosing of the AE which is preferably adjusted appropriately for the site and volume of adipose tissue as tolerated by the patient.

Preferably, the protocol for weeks 2 through 6 is adjusted based on clinical examination, findings, and how the patient is tolerating the procedure for any given treatment zone and clinical outcome. The neck and face preferably has fewer required treatments, but again will preferably be adjusted based on the site and volume of the adipose tissue as well as how the patient tolerates the procedure.

In yet another preferred embodiment, clinical judgment decides the choice of chemical, volume, spacing, time interval and proper use of the light base tightening unit as well as the RF unit based on clinical findings on examination.

Clinical judgment preferably looks at the following factors when determining a protocol or treatment regimen. These factors include, but are not limited to: (1) site and volume of the adipose tissue; (2) how the patient tolerates the procedure; (3) possible side effects; and (4) choice of chemical to be injected.

Although the invention has been described in detail with particular reference to these preferred embodiments, other embodiments can achieve the same results. Variations and modifications of the present invention will be obvious to those skilled in the art and it is intended to cover all such modifications and equivalents.

What is claimed is:

1. A method for destroying fatty tissue comprising: receiving mono-polar radio-frequency destruction treatment; administering an injection of a lipo-lysis agent into fatty tissue at least once; and performing a skin tightening procedure on the injection site.

2. The method of claim 1 wherein said mono-polar radio-frequency destruction treatment raises the temperature of the intended treatment area to at least 43° C. for approximately 2 minutes.

3. The method of claim 1 further comprising ultrasounding the treated area to actively diffuse the lipo-lysis agent within the fatty tissue.

4. The method of claim 3 wherein said ultrasounding is performed at 1 to 3 Hertz for at least 10 minutes.

5. The method of claim 1 wherein said injection of lypo-lysis agent is administered every 14 days.

6. The method of claim 1 wherein the injecting of lipo-lysis agent is 0.8 to 1.0 cc into fatty tissue.

7. The method of claim 1 wherein the injecting of lipo-lysis agent is at least 2 cm away from the previous injection.

8. The method of claim 1 wherein the skin tightening procedure uses bi-polar radio-frequency.

9. The method of claim 1 wherein the skin tightening procedure uses broadband light.

10. The method of claim 1 wherein the skin tightening procedure uses both bi-polar radio-frequency and broadband light.

11. The method of claim 1 wherein said lypo-lysis agent is an adipose emulsifier.

12. The method of claim 1 further comprising massaging the treated area to actively diffuse the lipo-lysis agent within the fatty tissue.
